(19) United States
(12) Patent Application Publication
(10) Pub. No.: US 2011/0287079 A1
(43) Pub. Date: Nov. 24, 2011

(54) SUPPORTING MATERIAL FOR CELL SHEET

(75) Inventors: Gonzalo Serafica, Newtown, PA (US); Constance Ace, Whitehouse, NJ (US); Jessica Donlin, Collegeville, PA (US); Junping Wang, Langhorne, PA (US)

(73) Assignee: Xylos Corporation

(21) Appl. No.: 13/022,124

(22) Filed: Feb. 7, 2011

Related U.S. Application Data
(60) Provisional application No. 61/346,198, filed on May 19, 2010.

(51) Int. Cl. A61K 9/00; A61K 35/12; A61P 17/00; A61P 27/02; A61P 19/04; A61P 9/00; C12N 5/071; C12P 19/04

(52) U.S. Cl. 424/423; 435/395; 424/93.7; 435/101

(57) ABSTRACT

Provided in one embodiment is an implantable support material for culturing cells, wherein at least some of the cells substantially maintain at least one of (i) phenotype and (ii) genotype thereof after being cultured on the support material.

### CROSS-REFERENCE TO RELATED PATENT APPLICATIONS

This application claims priority from U.S. Provisional Application No. 61/346,198, filed May 19, 2010, incorporated herein by reference in its entirety.

### FIELD OF INVENTION

All of the references, including any publications, patents or patent applications, cited in this Specification are incorporated herein by reference in their entirety.

### BACKGROUND

Cell sheet engineering is one of the newly developed concepts of tissue engineering in the last decade. The concept of culturing autologous cells ex vivo into confluent sheets prior to implant them has been demonstrated and well reviewed by Okano et el. (2009). The development of this technique started in the discovery of thermo-responsive polymers that can control attachment and detachment of cultured cells first reported by Yamada and Okano (1990). Since then, they have used polymer such as poly(N-isopropylacrylamide (PiPAAm) in culturing various cells sheets successfully. Other groups have attempted to create tissue engineered cellular sheets using various materials. In a recent patent by McAllister (U.S. Pat. No. 7,504,258), the investigators claim a method of producing a living stent, comprised of cells and extracellular matrix formed by the cells. Another group by Sanders also obtained a patent (U.S. Pat. No. 7,622,299) on bioengineering tissue substitutes using microfiber arrays. The fibers comprise biodegradable polymers such as poly lactic acid, poly caprolactone, poly glycolic acid, and poly urethanes as examples. Other materials have been used as support for culturing cell sheets; see Kikuchi (2005), in which various physicochemical characteristics of materials that can affect cell-material interaction were identified. Hydrophilic non-ionic polymers that are non cell-adhesive include polyethylene glycol, poly acrylamide, and polyvinyl alcohol. To date, the use of microbial cellulose as a viable support for cell sheet tissue engineering has not been reported.

Microbial cellulose has been demonstrated for culturing mammalian cells as early as 1993 by Watanabe et al., which showed the need to incorporate collagen to promote cell adhesion and achieve viable cell cultures for about 1 month. Their research was not focused on using microbial cellulose as viable support for cell sheet engineering, to produce confluent cell layers. Microbial cellulose combined with various polymers as implants were also attempted by Yasuda (2005) using the material in combination of poly acrylamide and gelatin. A patent application combining dissolve microbial cellulose sheets with polyvinyl alcohol were also reported by Wan (U.S. 2005/0037082). Most recently, a patent was granted on the use of microbial cellulose in contact lenses (U.S. Pat. No. 7,832,857). The patent adequately describes curved contact lenses comprising of microbial cellulose from 5% to 35% wt capable of correcting defects in vision. Additional desirable properties such as air permeability, light absorption were further claimed in the patent. An illustrative example of dissolving microbial cellulose in a solvent and subsequently precipitating in distilled water to obtain the lens was also described. However, none of these previous publications reported the use of microbial cellulose as viable support for cell sheet engineering with adequate strength transport cells and be sutured in place at the implantation site.

Thus, a need exists to fabricate a microbial cellulose based viable support for forming cell sheets, which cellulose should have desirable properties for optimal performance.

### BRIEF DESCRIPTION OF THE DRAWINGS

FIG. 1 shows light transmittance of ultra thin membrane, MTA, Vessel Guard, Securian within the visible spectrum (400-700 nm).

FIG. 2 shows results from a thickness and mechanical strength measurement for an embodiment of the ultra thin membrane.

FIG. 3 shows the results of a gene expression study of equine chondrocytes grown on one embodiment of Xylos microbial cellulose. Label “1” represents results from chondrocytes with biocellulose “disc; “2” cultured chondrocytes (no scaffold); “3” uncultured chondrocytes”; and “4” represents results from negative control.

FIGS. 4A-4B show: ARPE cells (A) on an ultra thin membrane of biocellulose of one presently described embodiment and (B) on plastic control plate. Both photos were taken after 10-days of culture. Cells are marked with GFP.

FIG. 5 shows the same cells at 28 days culture. Viable cells appear green while dead cells appear red.

FIG. 6 shows genetically modified cells in culture on the ultra thin membranes. Conditions were similar to those in FIG. 4 above.

FIGS. 7A-7C shows the application of ultra thin membranes as a subscleral implant.

FIG. 8 shows the histological response of the implants shown in FIGS. 7A-7C.

FIG. 9 shows conformability of microbial cellulose thin sheets over the cornea.

FIG. 10 shows the diffusion of a small molecule marker dye through the ultrathin membrane, compared to that of a collagen membrane currently used for similar applications.

### SUMMARY

One object of this invention relates to the use of microbial cellulose having suitable physical and chemical properties for use as supporting materials for fabricating cell sheets. The support material can be used for culturing mammalian cells by allowing the cells to grow to confluency and form sheets. The material can also enhance survival of the cells. Various types of cells can be cultured on the material while maintaining their respective phenotypes, genotypes, and/or morphology. Properties, such transparency, fluid holding capacity, strength, cell adhesiveness and conformability can be optimized. The material can also help the transfer of such cell sheets to the implant site by providing adequate support and can be easily removed without changing conditions (e.g., temperature) due to its minimal cell adhesion characteristics. The material can be made in various thicknesses and degrees of transparency as well as in resorbable and non resorbable forms. The water absorbency and conformability of the material can be controlled to optimize cell growth. Methods of making such a support material are also described.

One embodiment provides an implantable support material for culturing cells and that is capable of maintaining and transporting viable cell sheets, wherein at least some of the cells substantially maintain at least one of (i) phenotype and (ii) genotype thereof after being cultured on the support material.

In an alternative embodiment, a method of forming an implantable microbial cellulose support material for culturing cells is provided, the method comprising: (i) providing a microbial cellulose material, which has been fermented in a bioreactor for less than about 5 days; (ii) cleaning the microbial cellulose material; and (iii) pressing mechanically the microbial cellulose material, whereby the implantable microbial biocellulose support material is formed.

Another embodiment provides an implant material to be implanted into a subject in need thereof, the material comprising: (i) a microbial cellulose support material with adequate strength for the transfer of the cells and to be sutured in place; and (ii) a cell sheet disposed on the support material.

### DETAILED DESCRIPTION

The following detailed description illustrates specific embodiments of the invention, but is not meant to limit the scope of the invention. Unless otherwise specified, the words “a” or “an” as used herein mean “one or more.” The terms “substantially” and “about” used throughout this Specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

### Support Material

The support material that is to be implanted into a subject can have certain desirable properties, depending on the application. Some of these properties of cell sheet supports (“support material” or “cell support”) include biocompatibility, strength, conformability, and minimal cell-support material interaction. The biocompatibility can be important in allowing the cells to proliferate and form sheets containing viable cells. Depending on the application, mechanical strength of the cell support can be important in transporting the cell sheet to the intended implantation site, as well as the ability to manipulate the fragile sheet into place due to its conformability. Cell-material interaction can also be important especially in the detachment of the cell sheet from the support material after delivery to the implant site. Microbial cellulose has been found to be an effective support for cell sheet engineering and will be demonstrated in the examples below.

The support material can be a microbial cellulose-based material, such as one comprising a microbial cellulose produced by Acetobacter Xylinum. It is desirable to have at least some of the cells being cultured on the support material to retain their phenotype and/or genotype after being cultured on the support material. In one embodiment, substantially all of the cells retain their phenotype and/or genotype after being cultured. The cells can also retain their morphology. The cells can be any type of cell, depending on the applications. For example, the cells can be mammalian cells. The cells can be chondrocytes, synovial cells, epithelial cells, retinal pigment cells or combinations thereof.

The implantable material can be formed by any suitable methods. For example, microbial cellulose can be fermented in a bioreactor for a short period of time to form a thin film. Dependent on the desired properties of the film such as transparency, fluid holding capacity, strength and conformability, the time the fermentation process is allowed to progress may be varied. The microbial cellulose film will continue to grow (i.e. become thicker) as time progresses in the presence of adequate conditions. Thicker films have higher fluid holding capacity and strength whereas thinner films have more transparency and higher conformability. In one embodiment, in contrast to some of the presently existing biocellulose material, the presently described support material needs only a short period of fermentation time, which can be much less than one month. For example, the fermentation time can be less than about 10 days, such as less than about 5 days, such as less than about 2 days, such as less than about 1 day, such as less than about 20 hours, such as less than about 10 hours, such as less than about 5 hours. The fermented cellulose material can be cleaned to remove undesirable pyrogenic material. In one embodiment, the cellulose material is further mechanically pressed to remove a certain amount of liquid. The fabricated implantable support material can be packaged before being shipped to the consumer. Optionally, the microbial cellulose can be oxidized such as, for example, described in U.S. Pat. Nos. 7,645,874 and 7,709,631.

In one embodiment, the present described implantable support material can promote at least some of the cells, such as substantially all of the cells, being cultured to grow to confluency, or “form confluence.” For instance, in one embodiment, the cultured cells form a cell sheet. A cell sheet can be one disposed on a portion of the support material or cover substantially the entire surface of the support material. The presently described support can also enhance the survival or viability of the cells being cultured thereon/thereover. For example, substantially all of the cells being cultured can remain viable and subsequently grow to confluency. In one embodiment, the support material can have very low cell-adhesion characteristics. Specifically the cells only minimally interact with the support material. As a result, the support material can easily be removed from an implantation site or from the laboratory to be transferred to an implantation site.

In one embodiment, the support material can be relatively transparent. For example, the material can have a white light transmittance of at least about 50%, such as at least about 60%, such as at least about 70%, such as at least about 80%, such as at least about 85%, such as at least about 87%, such as at least about 90%, such as at least about 95%.

Depending on the application, the support material can have various thicknesses. For example, it can have a thickness of less than about 100 microns, such as less than about 90 microns, such as less than about 80 microns, such as less than about 70 microns, such as less than about 60 microns, such as less than about 50 microns, such as less than about 40 microns.

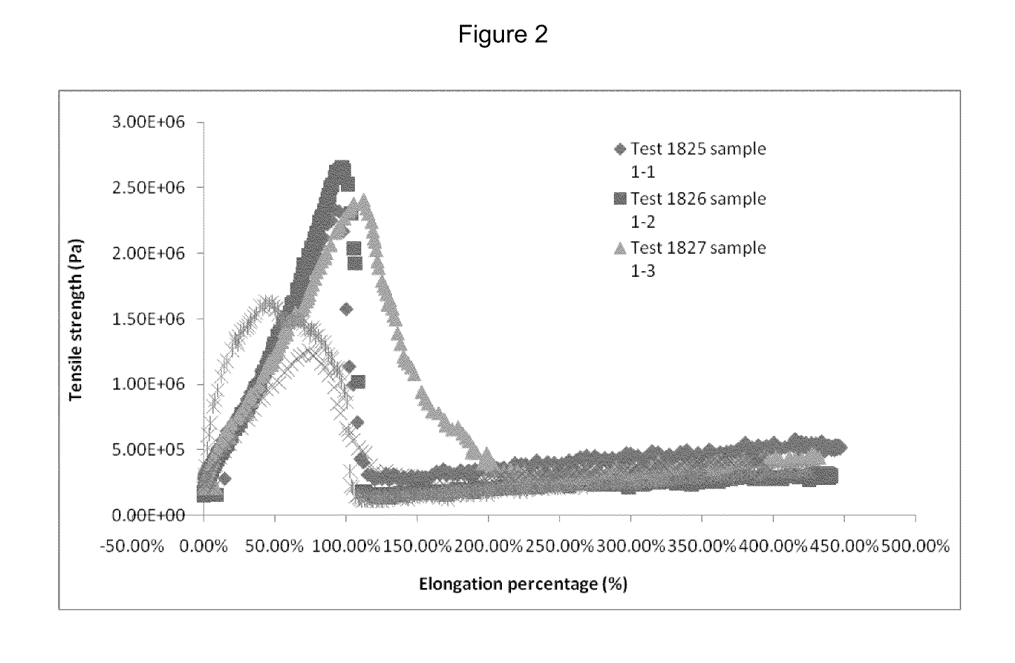

The support material can have different physical or mechanical properties, depending on the application. For example, the support material can have a tensile strength of at least about 1 MPa, such as at least about 1.5 MPa, such as at least about 2 MPa, such as at least about 2.5 MPa, such as at least about 3 MPa, such as at least about 3.5 MPa, such as at least about 4 MPa. In one embodiment, the support material has an elongation at break of at least about 40%, such as at least about 50%, such as at least about 60%, such as at least about 70%, such as at least about 80%, such as at least about 90%, such as at least about 95%, such as at least about 100%, such as at least about 110%, such as at least about 115%, such as at least about 120%. The support material can also have various elastic moduli, herein defined as the slope of the curve from the linear ramp-up region in a strength-elongation percentage curve, such as one shown in FIG. 2. The elastic modulus can be at least about 1 MPa, such at least about 1.5 MPa, such as about 2 MPa, such as about 2.5 MPa.

In another embodiment, adequate strength for the materials for use as support to be sutured in place and keep the transferred cell sheet viable until the graft is incorporated by the host. The key property of supporting viability is important for the transfer of cell sheets. The support material does not need to promote proliferation but on keep the cells viable and of the right phenotype and genotype.

It can be desirable to have the support material be as conformable as possible, especially if the site of implantation has a non-planar surface. Conformability can be measured by determining the angle of deflection of the material when held in a fixture that allows the material to extend out from a horizontal platform. For example, the angle formed between the material as it drapes off the end of the support and the support material can be measured. Values approaching 90° are indicative of highly conformable materials capable of applications where the material should conform to highly irregular or tightly curved surfaces. Values approaching 0° indicate stiff materials, which do not bend or conform under their own weight. These materials are more appropriate for use in applications where the surface to which they are applied is more planar. In one embodiment, the support material has a conformability of at least about 70°, such as at least about 80°, such as at least about 85°, such as at least about 88°, such as about 90°.

### Applications

The support material, together with a cell sheet as formed by the cultured cells on the support material, can be used as an implant material implanted into a subject in need thereof. The cells can form cell sheets over or on and not within, the support material. While some minute number of cells might be present in the support material, the presently described support material, which can be in the form of an ultra-thin membrane, can promote cells to form cell sheet thereon/there over. The support material and/or the implant as a whole can be bioresorbable or non-bioresorbable. The implant material can comprise a microbial cellulose support material and a cell sheet disposed thereon. The condition under which the subject needs to have the implant material can be a variety of conditions. For example, the implant can be used for any soft tissue repair, such as cornea repair, cartilage repair, connective tissue repair, heart tissue repair, ligament repair, dura tissue repair, or a combination thereof. The term “repair” herein can refer to replacement of entire tissue or a portion of a tissue, or using the implant as a supplement to the injured tissue, such as a patch or scaffold thereon to provide tissue regeneration.

Another embodiment is the ability of the said sheet to be used to maintain the cell sheet without promoting rapid proliferation. The key property of supporting viability is important for the transfer of cell sheets and subsequent take of the graft by the host. The support material does not need to promote proliferation during the healing period but only keep the cells viable and of the right phenotype and genotype.

### NON-LIMITING WORKING EXAMPLES

### Example 1

### Microbial Cellulose Fabrication

### Microbial Cellulose Preparation

To prepare the microbial cellulose for this invention, Acetobacter Xylinum microorganisms were cultured in a bioreactor containing a liquid nutrient medium at 30 degrees Celsius at an initial pH of 3-6. The medium was based on sucrose or other carbohydrates.

The bioreactor comprised a vessel with an open top. Dimensions of the bioreactor can be varied based on the area of support material needed. The open top of the bioreactor is covered but not sealed to limit contamination but allow for proper oxygen tension to be achieved.

The fermentation process under static condition was allowed to progress for a range of 5 hours to 5 days. During the fermentation process, the bacteria in the culture medium produced an intact cellulose film at the surface of the media. Excess media was needed to ensure the growth of the film was even across the surface of the media but not inhibited by the depth of the vessel. Fermentation was stopped by removing the film from the media.

### Cleaning Processing Procedures

The excess medium contained in the films was removed by chemical cleaning and subsequent processing. The cellulose film was subjected to a series of chemical washing steps to convert the raw cellulose film into a medical grade and non-pyrogenic film with desired transparency. Chemical processing started with treatment of the biocellulose with a 2% sodium hydroxide solution at 70-75° C. for 1 hour, followed by a series of rinses in de-ionized water. This was followed by a soak in 0.25% hydrogen peroxide for 1 hour then an overnight static rinse in de-ionized water.

### Optional Oxidation

Resorbable version of these sheets can be formulated using a similar starting material that has been oxidized. Varying levels of oxidation can render the material to lose mechanical integrity from weeks to several months. Full degradation of the these resorbable support sheets can be estimated to be in terms of months to years depending on the location of the implant, the amount of material and its degree of oxidation.

### Final Product Processing

Once cleaned and processed, the films were placed individually between two sheets of polyethylene terephthalate (PET). The films, once positioned between the PET, were subjected to mechanical pressing to remove excess water and decrease thickness.

Films were packaged between PET sheets in dual foil pouches and sterilized by gamma irradiation at 12-35 kGy.

### Example 2

### Physical Property Characterization

### Optical Properties of Support Material for Cell Sheet Fabrication

Light transmission of the ultra thin membrane was measured using a microplate reader (BioTeK) at 25° C. Four ultra thin membrane samples were tested (n=4). In addition, three other types of medical devices, Xylos MTA® protective sheet, vessel guard and Securian®, were also tested as negative controls. Each of the control groups contained three samples (n=3). Specifically, the ultra thin membrane (2.9 mm in diameter) was equilibrated in 1 ml phosphate buffered saline (PBS) in a 12 well-plate. A spectral scan from 400 nm to 700 nm (visible spectrum) was conducted at a resolution of 2 nm. The absorbance value was then converted to percent light transmission based on Beer's law, using Equation (1):

Absorbance=−log(percent transmittance/100) (1)

### Water Content and Thickness Measurement of the Ultra Thin Membrane

The water content of the biocellulose-based ultra thin membrane was determined by measuring the dry weight and wet weight of the samples. Four samples were used (n=4). For this study, wet ultra thin membrane was dried within the oven overnight under 60° C. after measuring the wet weight. The water content was calculated based on Equation (2)

Water content=1−Dry weight/Wet weight (2)

The thickness of the ultra thin membrane was measured through the observance of the cross section using an Olympus BX41TF Light Microscope equipped with Olympus DP20 Digital Camera and control box under 10× magnification. Briefly, the ultra thin membrane packed in two pieces of PET cover sheets were cut into strips (5 mm×30 mm). The strip was then fixed by using two glass slides, leaving the cross section of the membrane exposed within the observing field. Images were taken and processed using DP2-BSW 2.1 Software. The thickness of the membrane was calculated by averaging the measurement of the width of the cross section. Five samples were tested (n=5).

### Tensile Properties of the Ultra Thin Membrane

The mechanical properties (tensile strength and elongation at rupture) of 50 μm thick rectangular ultrathin membrane strip (10 mm×40 mm) were determined using a tensile tester SSTM 2KM (United Testing Systems, Inc.) at a speed of 3 mm/sec with a preload of 0.1 N. Five samples were tested (n=5).

### Conformability of the Ultra Thin Membrane

The conformability of the ultra-thin membrane was measured by determining the angle of deflection of the material when held in a fixture that allowed the material to extend out from a horizontal platform. The angle formed between the material as it draped off the end of the support and the support material was measured and compared to that of the negative control.

### Results

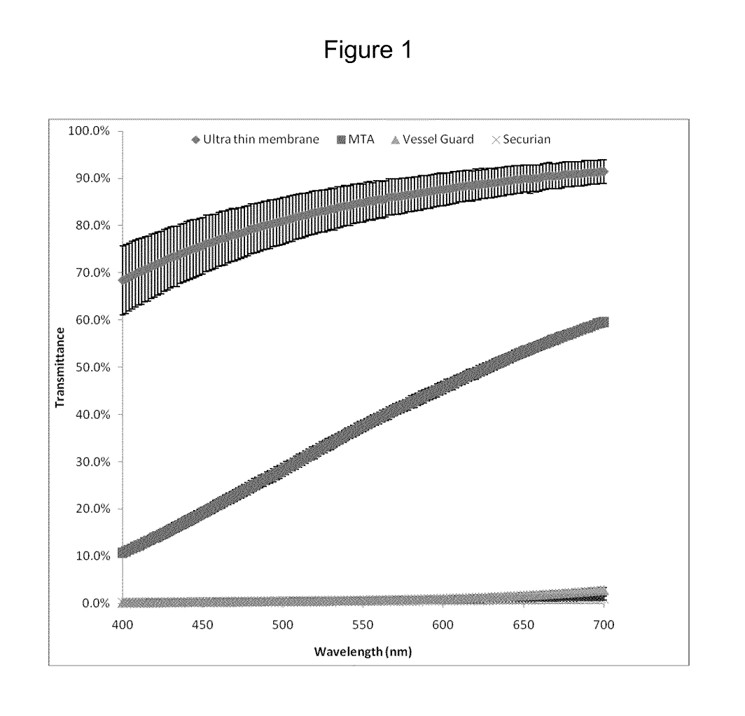

The light transmittance within the visible spectrum (400 nm-700 nm) is shown in FIG. 1. The white light transmittance was 83.3%±4.3%. This value is very close to the light transmittance of human cornea 87% [1] and has a similar spectral absorbance distribution, indicating that Xylos® ultra thin membrane can be an optimal material for potential human corneal application. The white light transmittance for the other three types of medical devices, which served as the negative controls, were 36.5%±1.2% (MTA), 1.0%%±0.1% (Vessel Guard), 0.5%±0.1% (Securian). These data confirm that the subject membranes of microbial cellulose can provide optimal support for cultured ophthalmic cells while other membranes of similar composition do not provide sufficient transparency for that purpose.

The average water content of the ultra thin membrane was 96.95%±0.63%. The measured physical and mechanical properties are provided in the table below, and some of the data are also shown in FIG. 2. The average thickness of the ultra thin membrane was 45.8±11.0 μm. The tensile strength of the ultra thin membrane was 2.06±0.58 MPa, with the elongation at break being 85±25%; the elastic modulus was 1.97±0.27 MPa. The data are comparable to the mechanical properties of the human cornea which has tensile strength: 3.81±0.40 MPa [5]; elongation at break: 60.0±15.0% [2]; and modulus: 3-13 MPa [3]. Furthermore, the ultra thin membrane made from biocellulose sheet described herein is much stronger than the currently existing collagen based products, which have tensile strength at (0.5-0.8 MPa), modulus (1-1.5 MPa) [4].

| | Thickness | Tensile strength | Elongation | Elastic Modulus |
|---|---|---|---|---|
| sample | (μm) | (MPa) | at break | (MPa) |
| 1-1 | 30.7 | 2.33 | 92% | 2.01 |
| 1-2 | 61.5 | 2.65 | 97% | 2.20 |
| 1-3 | 43.8 | 2.41 | 112% | 1.91 |
| 1-4 | 46.6 | 1.26 | 74% | 1.55 |
| 1-5 | 46.2 | 1.63 | 48% | 2.19 |
| Ave | 45.8 | 2.06 | 85% | 1.97 |
| Stdev | 11.0 | 0.58 | 25% | 0.27 |

### Conformability of the Ultra Thin Membrane

The conformability of the Ultra Thin Membrane was measured and compared to the conformability of two negative controls. These data are shown in the following table:

| Sample | Securian ™ | MTA ™ | Ultra-thin membrane |
|---|---|---|---|
| 1 | 0 | 68 | 90 |
| 2 | 2 | 76 | 88 |
| 3 | 0 | 58 | 90 |
| 4 | −1 | 62 | 90 |
| 5 | 0 | 61 | 88 |
| Average | 0.2 | 65.0 | 89.2 |
| Stdev | 1.1 | 7.1 | 1.1 |

These data show that the ultra thin membrane specimens were highly comfortable, and thus can be used in cell sheet applications where the surface to which the cells are to be applied is irregular or has a non-planar surface.

### Example 3

### Biological Properties Characterization

### Cell Culture on Biocellulose Membranes

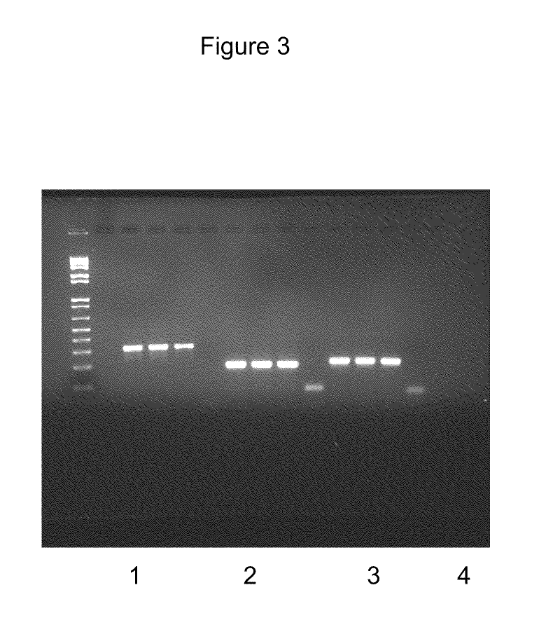

The microbial cellulose described herein has been shown to be an optimal growth matrix for cell proliferation, as well as cell vitality. A common problem with many cell growth matrices is that they will support cell growth but at the same time allow or drive cell de-differentiation. Examples include culture of chondrocytes on conventional support matrices, where the cells in culture morph into less-differentiated cells such as fibroblasts, losing their ability to express the genes characteristic of the more highly differentiated chondrocytes. This study shows that chondrocyte cells grown on the presently described microbial cellulose membranes proliferate better than those grown without the microbial cellulose present. Results from a gene expression study of these cells are shown in FIG. 3.

The expression of Aggrecan and Collagen Type II confirm that the cells after culture have retained their genotypic protein expression characteristics. These proteins would not be expressed if the cells had de-differentiated to the more generic cell type, fibroblasts. Separate studies confirmed the absence of collagen type I expression characteristic of fibroblasts.

Similar results have been shown for human chondrocytes and neonatal porcine chondrocytes as well as equine synovial cells. In additional studies, human synovial fibroblasts showed gene expression that was phenotypically characteristic for synovial cells, showing expression levels for type I collagen, as well as biglycan and decorin.

Together these studies confirm the ability of the presently described microbial cellulose membranes to support robust cell growth without driving de-differentiation of the cells. Such de-differentiation would lead to an undesirable loss of functional properties for the resultant cell sheet.

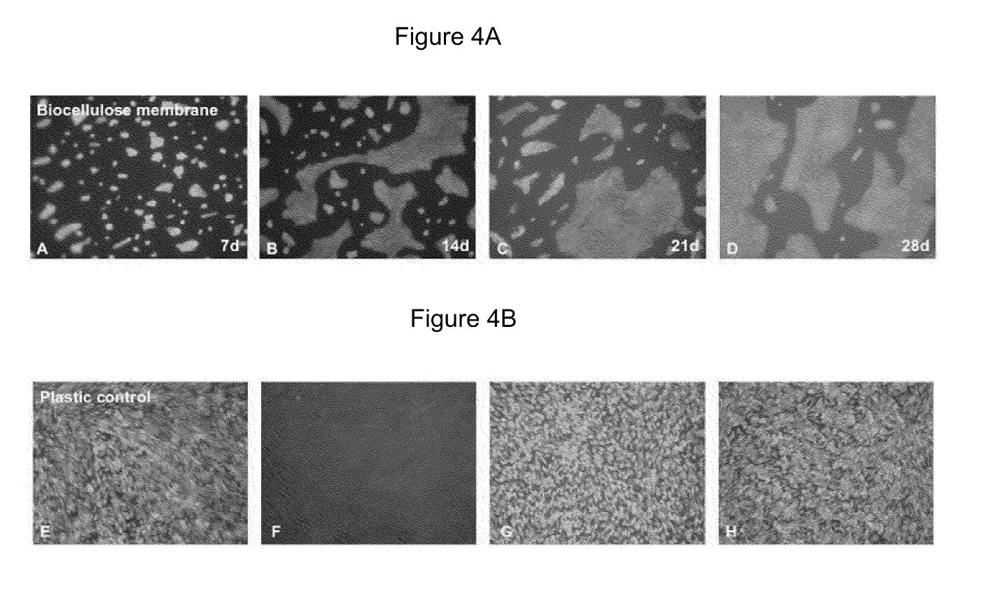

Cell culture studies have also confirmed the vitality of various ophthalmic cells grown on Xylos biocellulose membranes. FIG. 4A shows the proliferation of retinal pigment cells ARPE-19 on an ultra thin membrane and FIG. 4B on plastic control. Partial confluency is established in FIG. 4A. Compared to the plastic control substrate, cell mortality is significantly reduced These photos demonstrate the cells' ability to proliferate on the membrane and the membrane's ability to provide greater cell survival over extended periods.

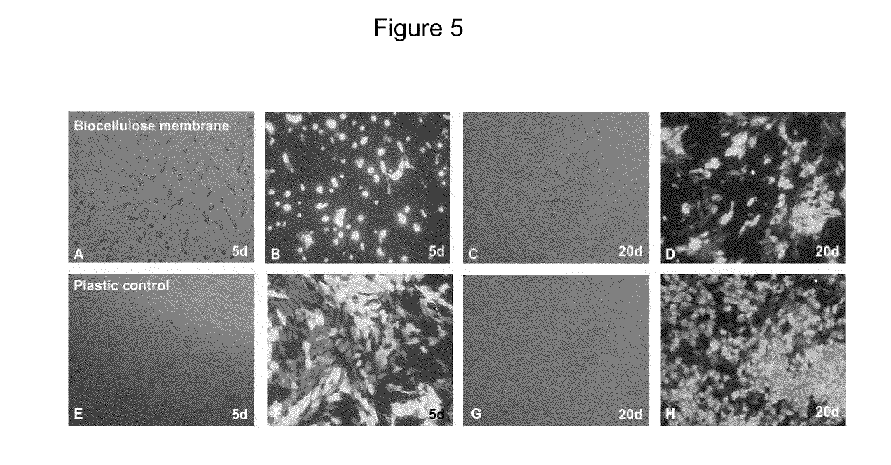

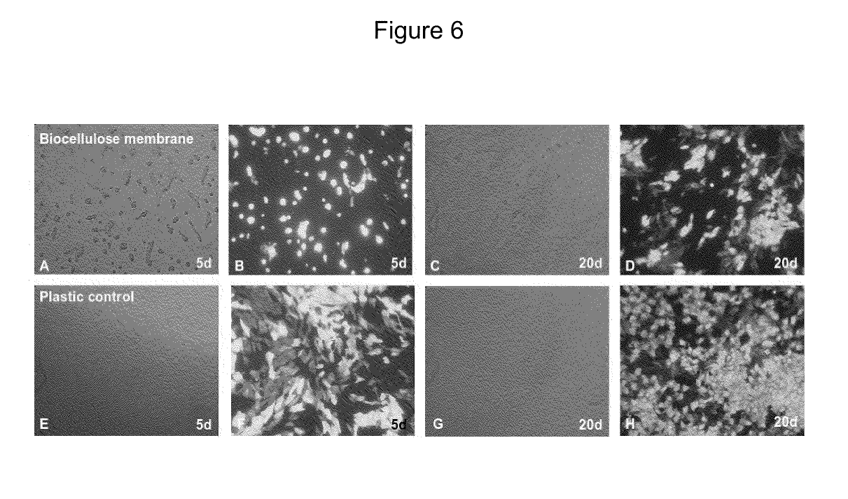

FIG. 5 shows the ability of the cell support membrane to provide sustained viability of the cells with minimal cell mortality. FIG. 6 shows the ability of the membrane to sustain viability of genetically engineered cells. The ability to support either native or engineered cells is an important feature where these cells need to be sustained in culture, with minimal cell mortality, prior to transplantation.

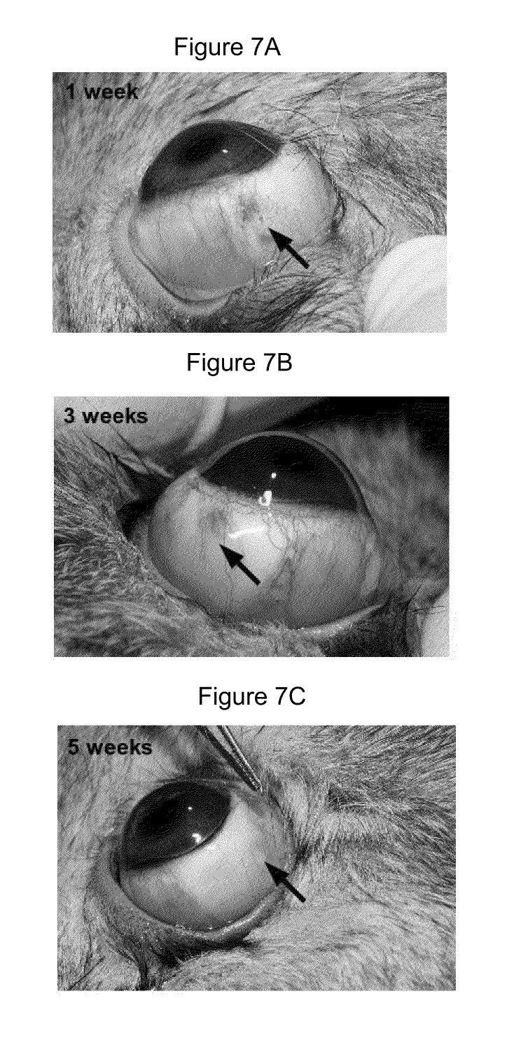

The cell support membranes show were implanted into the eye to assess their biocompatibility as an ophthalmic implant. FIGS. 7A-7C show the subscleral implant site. Most notable in these photos is the absence of adverse tissue reaction to the implant. Irritation which is observed is generally associated exclusively with the ophthalmic suture used to affix the implant, and not with the implant itself. These photos demonstrate that the membranes have utility as an ophthalmic implant, either with or without cultured cells.

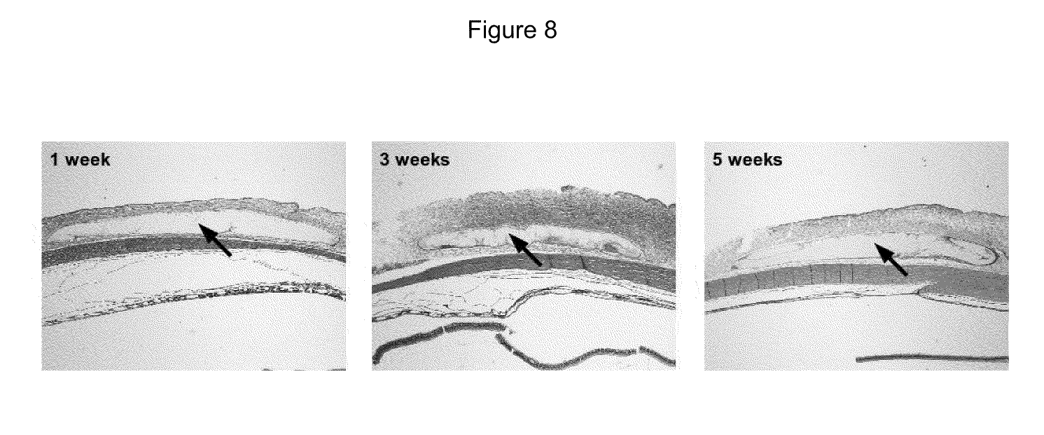

FIG. 8 shows the histological response of the surrounding tissues to the implants shown in FIGS. 7A-7C. The lack of significant inflammatory response or scar tissue shown in these photomicrographs further supports the utility of the device as an implantable cell support sheet.

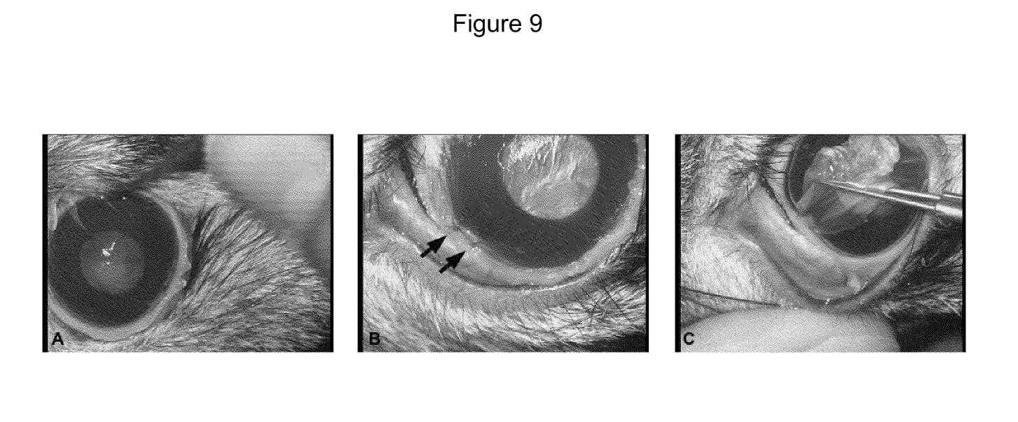

One application of cell sheet transfer is for repair of defects in and around the cornea. To assess the membranes for this application, the membranes were applied to the eye in a rabbit model. FIG. 9 shows the conformability of the membrane in that it is able to conform to the acute curvature of the rabbit eye without bucking or folding. These results further support the utility of the membranes as a cell support sheet for use in areas where conformability is critical.

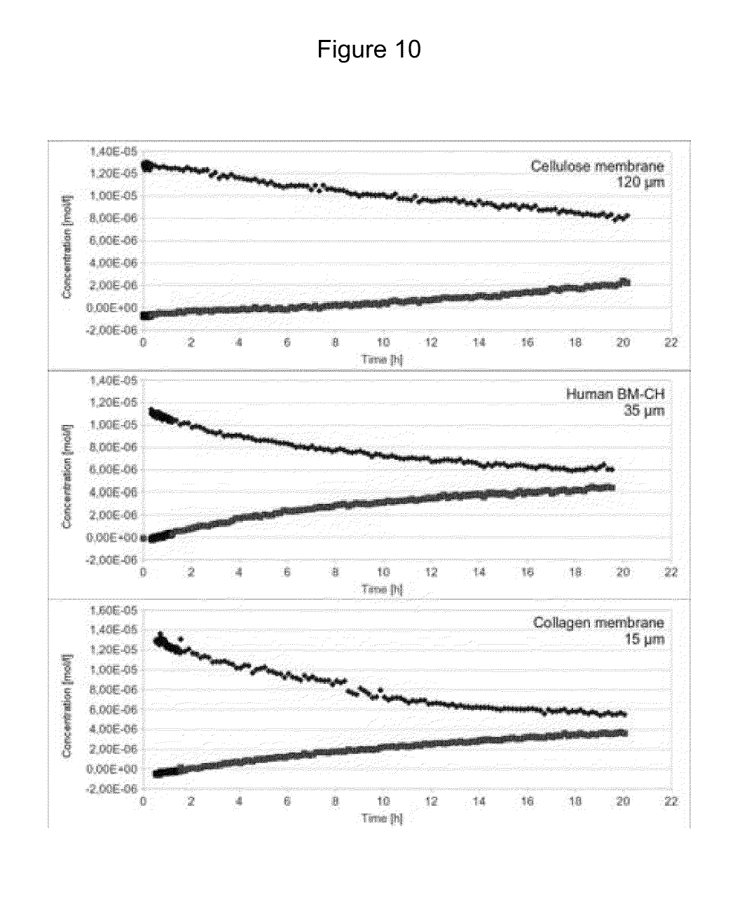

The ability of a cell support membrane to allow free diffusion of fluid is important for ophthalmic applications. FIG. 10 compares the diffusivity of a small marker molecule, bromothymol blue, through the membrane. Diffusivity was shown to be similar to that of a thin collagen membrane currently used for similar applications. This data show that the membrane is sufficiently permeable to allow facile diffusion of fluid and thus allow nutritive support of cells on the membrane.

The degradation of these cellulose support sheets can vary from permanent/non degrading to degradable/resorbable sheets at varying rates. Depending on the level of chemical modification, these resorbable versions can degrade after implantation from days to months and even years. However, it is more likely that resorbable sheets for this application to have mechanical integrity for days and up to the time the cell sheets themselves have gained full integrity and fully regenerated. This can be anywhere from two weeks to six months.

### REFERENCES

[1] Beems E M, Best J V. Light transmission of the cornea in whole human eyes. Exp Eye Res 1990; 50:393-5.
[2] Zeng Y, Yang J, Huang K, Lee Z, Lee X. A comparison of biomechanical properties between human and porcine cornea. J Biomech 2001; 34: 533-7.
[3] Rafat M, Li F, Fagerholm P, Lagali N S, Watsky M A, Munger R, et al. PEG stabilized carbodiimide crosslinked collagen-chitosan hydrogels for corneal tissue engineering. Biomaterials 2008; 29(29):3960-72
[4] Crabb R A, Chau E P, Evans M C, Barocas V H, Hubel A. Biomechanical and microstructural characteristics of a collagen film-based corneal stroma equivalent. Tissue Eng 2006; 12:1565-75.

What is claimed:

1. An implantable support material for culturing cells and that is capable of maintaining and transporting viable cell sheets,
wherein at least some of the cells substantially maintain at least one of (i) phenotype and (ii) genotype thereof after being cultured on the support material.

2. The implantable support material of claim 1, wherein the support material comprises microbial cellulose.

3. The implantable support material of claim 1, wherein the support material comprises oxidized microbial cellulose.

4. The implantable support material of claim 1, wherein the cells are mammalian cells.

5. The implantable support material of claim 1, wherein the cells are chondrocytes, synovial cells, epithelial cells, retinal pigment cells or combinations thereof.

6. The implantable support material of claim 1, wherein at least some of the cultured cells form at least partial confluence.

7. The implantable support material of claim 1, wherein at least some of the cultured cells form a cell sheet.

8. The implantable support material of claim 1, wherein the support material has a white light transmittance of at least about 80%.

9. The implantable support material of claim 1, wherein the support material has a thickness of less than about 60 microns.

10. The implantable support material of claim 1, wherein the support material has a tensile strength of at least about 1.5 MPa.

11. The implantable support material of claim 1, wherein the support material has an elongation at break of at least about 60%.

12. The implantable support material of claim 1, wherein the support material has a conformability of at least 88°.

13. The implantable support material of claim 1, wherein at least some of the cells form a cell sheet over the support material.

14. A method of forming an implantable microbial cellulose support material for culturing cells, comprising:
(i) providing a microbial cellulose material, which has been fermented in a bioreactor for less than about 5 days;
(ii) cleaning the microbial cellulose material; and
(iii) pressing mechanically the microbial cellulose material, whereby the implantable microbial cellulose support material is formed.

15. The method of claim 14, wherein the cleaning in step (ii) further comprises treating the microbial cellulose material with a sodium hydroxide solution.

16. The method of claim 14, wherein the microbial cellulose material is made by Acetobacter Xylinum.

17. The method of claim 14, further comprising packaging the implantable biocellulose support material after step (iii).

18. The method of claim 14, wherein the microbial cellulose material comprises oxidized microbial cellulose.

19. An implant material to be implanted into a subject in need thereof, the material comprising:
(i) a microbial cellulose support material with adequate strength for the transfer of the cells and to be sutured in place; and
(ii) a cell sheet disposed on the support material.

20. The implant material of claim 19, wherein the implant is for cornea repair, cartilage repair, connective tissue repair, heart tissue repair, ligament repair, dura tissue repair, or a combination thereof.

21. The implant material of claim 19, wherein the support material has a conformability of at least 88°.

22. The implant material of claim 19, wherein the support material has a white light transmittance of at least 80%.

23. The implantable material of claim 19, wherein substantially all of the cells in the cell sheet are viable.

24. The implantable material of claim 19, wherein the microbial cellulose material comprises oxidized microbial cellulose.
